(19) United States
(12) Patent Application Publication
(10) Pub. No.: US 2006/0115526 A1
(43) Pub. Date: Jun. 1, 2006

(54) Venlafaxine compositions comprising pellets with double layer coating

(76) Inventors: Chetan Chhabildas Doshi, Plainsboro, NJ (US); Dinesh Gopinathan, Maharastra (IN); Sushrut Kulkarni, Kalyan (IN); Mangesh Sukthankar, Maharastra (IN); Prashant Thakker, Maharashtra (IN)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/551,106

(22) PCT Filed: Mar. 26, 2004
(86) PCT No.: PCT/EP04/03255

§ 371 Date: Oct. 25, 2005

(30) Foreign Application Priority Data
Mar. 28, 2003 (GB) 0307277.4
Jul. 9, 2003 (GB) 0316087.6

(51) Int. Cl. A61K 9/64; A61K 9/26

(52) U.S. Cl. 424/456; 424/470

(57) ABSTRACT

This invention provides coated pellets comprising a) a pellet core which comprises venlafaxine hydrochloride; b) a first coating which comprises a lipophilic layer or a sparingly water-soluble layer, and c) a second coating which comprises a water-insoluble polymer or polymer mixture. In another aspect this invention provides compositions which comprises the coated pellets. In a further aspect, the invention provides a process for preparing the coated pellets which process avoids use of organic solvents at least for the second coating.

This invention relates to pharmaceutical compositions of venlafaxine.

Venlafaxine is the non-proprietary name for 1-[2-(dimethylamino)-1-(4-methoxyphenyl)ethyl]cyclohexanol and is useful in treating a number of disorders including depression, anxiety, panic disorder and pain. Venlafaxine is administered as venlafaxine hydrochloride in treating depression. See The Merck Index, 12th Edition, entry 10079.

Published European patent application EP 797 991 A discloses encapsulated extended release formulations of venlafaxine hydrochloride which comprise a hard gelatin capsule containing spheroids comprised of venlafaxine hydrochloride, microcrystalline cellulose and hydroxypropylmethylcellulose coated with ethyl cellulose and hydroxypropylmethylcellulose. Solvents used in the coating step include methylene chloride and anhydrous methanol.

The present applicants have sought to overcome the drawbacks of hitherto known formulations of venlafaxine.

In one aspect, therefore, this invention provides coated core pellets comprising venlafaxine for use in a delayed and/or extended release formulation which core pellets undergo at least one coating step in the absence or substantial absence of organic solvents.

In another aspect, this invention provides a coated pellet comprising

a) a pellet core which comprises venlafaxine hydrochloride;

b) a first coating which comprises a lipophilic layer or a sparingly water-soluble layer, and

c) a second coating which comprises a water-soluble or water-insoluble polymer.

The pellet core may comprise in addition a carrier, for example microcrystalline cellulose. The pellet core may comprise in addition a binder, for example a cellulose derivative, e.g. hydroxypropylmethylcellulose (HPMC). The present applicants have found that HPMC of e.g. grade K 4 M has an appropriate viscosity for use in this invention.

The pellet core may be spheroidal in geometry and typically exhibits a diameter, when coated, of between around 0.5 mm to 2 mm, e.g. 0.7 to 1.75mm, for example 0.8 mm to 1.5 mm.

The first coating and second coating may each be complete or substantially complete, e.g. so as to provide a surface coverage of at least 60%, e.g. 70% or more, e.g. 80 to 95% around the core (first coating) or around the first coating (second coating). Complete coatings are preferred.

The first coating may comprise between 0.5 to 5% by weight, e.g. 1 to 4%, of the first-coated pellet core.

The second coating may comprise 8 to 30% by weight, e.g. 10 to 25%, based on the total weight of the double-coated core.

The first coating serves to protect the pellet core from moisture, both in storage and in use.

The lipophilic layer may comprise a fat, fatty alcohol or wax. The lipophilic layer preferably comprises cetostearyl alcohol, castor oil or dibutyl phthalate.

The sparingly water-soluble layer may comprise a carbohydrate or a sugar, e.g. lactose, in the form of an aqueous suspension wherein the concentration of the carbohydrate or sugar is at least about 0.1 g/ml, e.g. 0.15 to 0.25 g/ml or greater, e.g. 0.28 g/ml to 0.4 g/ml or even higher, e.g. 0.41 to 0.6 g/ml, for example 0.43 or 0.5 g/ml.

The sparingly water-soluble layer, which may be formed by spray-coating, may comprise lactose in an amount of up to about 30% to 40% by weight.

The first coating may be free of, or substantially free of, ethyl cellulose.

The water-soluble or water-insoluble polymer may be selected from acrylate-based aqueous dispersions, ethylcellulose aqueous dispersions and polyvinyl acetate aqueous dispersions. Thus the second coating is aqueous-based and serves to provide the extended release effect.

Water-insoluble polymers are preferred and may serve to control release of the venlafaxine. The water-insoluble polymer may display pH-independent solubility and may comprise a water-insoluble polymer mixture.

The term “water-insoluble”, as used herein is understood to mean a polymer solubility in water at room temperature of less than 100 mg/litre, e.g. 20 mg/litre or less, e.g. 10 mg/litre or less, e.g. 1 mg/litre or less.

In a preferred aspect, the pellet core and/or coating(s) of this invention are free of, or substantially free of, polyvinylpyrrolidone.

In another aspect, this invention provides a composition comprising coated pellets as herein described. The composition may be in tablet, hard gelatine capsule or sachet form.

In another aspect, this invention provides a coated pellet consisting of or consisting essentially of

a) a core containing venlafaxine hydrochloride in an amount of between 30 and 60% by weight, microcrystalline cellulose in an amount of between 40 and 65% by weight, and BPMC K 4 M in an amount of between 0.3 and 0.8% by weight, wherein the respective weights are in relation to the double-coated core;

b) a first coating containing cetostearyl alcohol in an amount of between 1.0 and 4.0%, e.g. 1.7 and 3.5%, by weight of the first-coated pellet core; and

c) a second coating containing an acrylate-based polymer in an amount of between 9 and 25%, e.g. 9 and 13%, by weight based on the total weight of the double-coated core, and talc in an amount of between 2 and 15%, e.g. 3 and 8%, by weight based on the total weight of the double-coated pellet core.

Suitable acrylate-based polymers or water-insoluble polymers having pH-independent solubility are available commercially e.g. from the Rohm company, Germany, under the trade marks EUDRAGIT, SURELEASE or AQUACOAT, e.g. EUDRAGIT NE 30 D, EUDRAGIT RL 30 D, EUDRAGIT RS 30 D or KOLLCOAT SR as dry polymer.

The second coating may further comprise triethyl citrate or dibutyl phthalate, e.g. in an amount of 5 to 35%, e.g. 10 to 30%, by weight of the dry polymer.

In a further aspect, this invention provides a composition consisting of or consisting essentially of coated pellets as herein described. The composition may be in tablet, hard gelatine capsule or sachet form.

In a further aspect, this invention provides a process for preparing pellets as herein described which comprises the steps of

Ai) forming a pellet core mixture comprising venlafaxine hydrochloride with water or an aqueous solution of a binder,

Aii) extruding and spheronising the mixture, and subsequently drying,

Aiii) applying the first coating,

Aiv) applying the second coating, and subsequently sieving so as to obtain coated pellets within the desired size range,

wherein the process is carried out in the absence or substantial absence of any organic solvent at least in the second layer.

Coating steps Aiii) and Aiv) may employ conventional fluidised bed processes Alternatively, the first coating layer may be applied using a spray melt process or by using a tangential coating process.

In another embodiment, the first coating layer may be dissolved in an organic solvent medium, e.g. methylene chloride or methanol, and sprayed onto the pellet cores.

In a further aspect, this invention provides a process for preparing pellets as herein described which comprises the steps of

Bi) forming a pellet core mixture comprising venlafaxine hydrochloride, microcrystalline cellulose and HPMC with water or an aqueous solution of a binder,

Bii) extruding and spheronising the mixture, and subsequently drying,

Biii) collecting the core pellets between 0.8 mm to 1.75 mm for further processing,

Biv) applying the first coating, and

Bv) applying the second coating

wherein the process is carried out in the absence or substantial absence of any organic solvent at least in the second layer.

Coating steps Biv) and Bv) may employ conventional fluidised bed processes. Alternatively, the first coating layer may be applied using a spray melt process or by using a tangential coating process.

A preferred embodiment of each of the above processes is such that the process is carried out in the absence or substantial absence of any organic solvent in both the first coating layer and in the second coating layer.

The venlafaxine hydrochloride is sourced from the Medichem company, Spain. The venlafaxine may be used in any polymorphic form, e.g. in the forms known as Form I or Form II. The compositions of this invention may be administered to adults in doses ranging from 75 mg to 350 mg venlafaxine per day.

Following is a description by way of example only of compositions of this invention.

### EXAMPLE 1

Pellets according to the following composition are prepared and filled into hard gelatin capsules.

| | | Quantity per capsule (mg) |
|---|---|---|
| I | Core pellets | |
| | venlafaxine HCl | 169.70 |
| | microcrystalline cellulose | 199.0 |
| | HPMC K 4 M | 1.85 |
| II | Wax coating | |
| | cetostearyl alcohol | 9.26 |
| III | Polymer coating | |
| | Eudragit NE 30 D | 56.97 |
| | talc | 28.49 |

| | |
|---|---|
| Total weight of coated pellets | 476.90 |

The core pellets are prepared by mixing the above components with a small amount of water, i.e. enough to form a paste without dissolving the venlafaxine, under I followed by extrusion spheronisation. The wax coating is applied using a fluidised bed process at or close to the melting temperature of the coating layer. The subsequent polymer (sustained release) coat is applied by a fluidised bed process. The resulting coated pellets are sieved so as to obtain a desired pellet size range of between 0.85 mm and 1.75 mm.

### EXAMPLE 2

Pellets are prepared in analogous manner to those in Example 1 with replacement of the wax coating by an aqueous suspension of lactose at a concentration of 0.15 g/ml. The suspension is sprayed onto the cores using a perforated pan or a fluidised bed process.

In view of the small amount of water involved in the first coating step, negligible dissolution of venlafaxine takes place and a protective layer is formed between the pellet core and the second coating.

### EXAMPLE 3a) AND 3b)

Pellets according to the following composition are prepared and filled into hard gelatin capsules.

| | | Quantity per capsule (mg) |
|---|---|---|
| I | Core pellets | |
| | venlafaxine HCl | 169.70 |
| | microcrystalline cellulose | 193.27 |
| | HPMC K 4 M | 1.85 |
| II | Wax coating | |
| | cetostearyl alcohol | 10.03 |
| III | Polymer coating | |
| | Eudragit NE 30 D | 70.10 |
| | talc | 35.05 |

| | |
|---|---|
| Total weight of coated pellets | 480.00 |

The core pellets are prepared by mixing the above components with a small amount of water, i.e. enough to form a paste without dissolving the venlafaxine, under I followed by extrusion spheronisation. The pellets with size between 0.8 mm and 1.75 mm are collected. The wax coating is applied using

in Example 3a) a fluidised bed process, and

in Example 3b) tangential coater,

at or close to the melting temperature of the coating layer. The subsequent polymer (sustained release) coat is applied by a fluidised bed process.

### EXAMPLES 4a) AND 4b)

Pellets are prepared in analogous manner to those in Example 1 with replacement of the wax coating by an aqueous suspension of lactose at a concentration of 0.43 g/ml. The suspension is sprayed onto the cores using

4a) a perforated pan, or

4b) a fluidised bed process;

In view of the small amount of water involved in the first coating step, negligible dissolution of venlafaxine takes place and a protective first layer is formed between the pellet core and the second coating.

### EXAMPLE 5

A capsule composition is prepared in analogous manner to that in Example 1 with the following component amounts.

| | | Quantity in % | |
|---|---|---|---|
| I | Core pellets | | |
| | venlafaxine HCl | 37.3% | by weight of core pellets |
| | microcrystalline cellulose | 62.1% | by weight of core pellets |
| | HPMC K 4 M | 0.5% | by weight of core pellets |
| II | Wax coating | | |
| | cetostearyl alcohol | 2.5% | by weight of core pellets |
| III | Polymer coating | | |
| | Eudragit NE 30 D (dry) | 15% | by weight of pellets with |
| | | | first coating |
| | talc | 50% | by weight of dry polymer |

The following dissolution profiles are observed using USP Apparatus 1 at 100 rpm in purified water at 37° C.

Cumulative amount dissolved (%)

| Time | | Formulation of | Dissolution |
|---|---|---|---|
| (hours) | EFFEXOR ER | Example 3 | range in % |
| 2 | 14 | 8 | <30 |
| 4 | 40 | 32 | 30 to 55 |
| 8 | 67 | 68 | 56 to 80 |
| 12 | 79 | 85 | 65 to 90 |
| 24 | 93 | 98 | >80 |

The principal advantages of the pellets and compositions of the present invention include a release profile of venlafaxine as effective as the commercially available product, however without the use of an organic solvent medium at least for application of the second coating. A further advantage is the absence of any organic solvent residue in the coated pellets.

The process is more cost-effective and less harzardous than hitherto known processes.

The coated pellets of this invention are thus produced using more economically and environmentally attractive processes than hitherto known processes for venlafaxine.

1. A coated pellet comprising
a) a pellet core which comprises venlafaxine hydrochloride;
b) a first coating which comprises a lipophilic layer or a sparingly water-soluble layer, and
c) a second coating which comprises a water-insoluble polymer or polymer mixture.

2. A pellet as claimed in claim 1 wherein the core additionally comprises microcrystalline cellulose.

3. A pellet as claimed in claim 1 wherein the core further comprises a binder.

4. A pellet as claimed in claim 3 wherein the binder comprises a cellulose derivative.

5. A pellet as claimed in claim 3 wherein the binder comprises hydroxypropyl methylcellulose.

6. A pellet as claimed in claim 1 wherein the lipophilic layer comprises a component selected from the group consisting of a fat, fatty alcohol and a wax.

7. A pellet as claimed in claim 1 wherein the lipophilic layer comprises a component selected from the group consisting of cetostearyl alcohol, castor oil and dibutyl phthalate.

8. A pellet as claimed in claim 1 wherein the sparingly water-soluble layer comprises a sugar, in the form of an aqueous suspension wherein the concentration of the sugar is at least 0.3 g/ml.

9. A pellet as claimed in claim 1 wherein the water-insoluble polymer is selected from the group consisting of polymethacrylate dispersions, ethylcellulose dispersions and polyvinyl acetate dispersions.

10. A composition comprising pellets as claimed in claim 1.

11. A composition as claimed in claim 10 in a form selected from the group consisting of a tablet, hard gelatine capsule and sachet.

12. A process for preparing coated pellet cores which process comprises the steps of
i) forming a pellet core mixture comprising venlafaxine hydrochloride with water or an aqueous solution of a binder,
ii) extruding and spheronising the mixture, drying and sieving,
iii) applying a first coating, and
iv) applying a second coating,
wherein the process is carried out in the substantial absence of any organic solvent medium in the second coating.

13. A process as claimed in claim 12 wherein the pellet core mixture further comprises microcrystalline cellulose and HPMC.

14. A process as claimed in claim 12 wherein the process is carried out in the substantial absence of any organic solvent medium in both the first and second coatings.

15. A coated pellet produced by the process as claimed in claim 12.

16. A coated pellet comprising
a) a core containing venlafaxine hydrochloride in an amount of between 30 and 60% by weight, microcrystalline cellulose in an amount of between 40 and 65% by weight, and HPMC K 4 M in an amount of between 0.3 and 0.8% by weight, wherein the respective weights are in relation to the double-coated core;
b) a first coating containing cetostearyl alcohol in an amount of between 1.7 and 3.5% by weight of the first-coated pellet core; and
c) a second coating containing an acrylate-based polymer in an amount of between 9 and 13% by weight based on the total weight of the double-coated core, and talc in an amount of between 3 and 8% by weight based on the total weight of the double-coated pellet core.

17. A composition comprising pellets as claimed in claim 16.
